(19) United States
(12) Patent Application Publication
(10) Pub. No.: US 2023/0160895 A1
(43) Pub. Date: May 25, 2023

(54) SARS CORONAVIRUS 2 DIAGNOSTIC KIT INCLUDING RECEPTOR AND ANTIBODY BINDING TO SARS CORONAVIRUS 2 SPIKE PROTEIN

(71) Applicant: KOREA RESEARCH INSTITUTE OF CHEMICAL TECHNOLOGY, Daejeon (KR)

(72) Inventors: Hong Gi KIM, Daejeon (KR); Jonghwan LEE, Daejeon (KR); Bum Tae KIM, Daejeon (KR)

(73) Assignee: KOREA RESEARCH INSTITUTE OF CHEMICAL TECHNOLOGY, Daejeon (KR)

(21) Appl. No.: 17/997,151

(22) PCT Filed: Jun. 18, 2021
(86) PCT No.: PCT/KR2021/007668

§ 371 Date: Oct. 26, 2022

(30) Foreign Application Priority Data
Apr. 29, 2020 (KR) 10-2020-0052619
Nov. 11, 2020 (KR) 10-2020-0149886

(51) Int. Cl. G01N 33/569; G01N 33/543

(52) U.S. Cl. CPC G01N 33/56983; G01N 33/54346; G01N 2333/165; G01N 2333/948

(57) ABSTRACT

Provided are a composition for detecting SARS coronavirus 2, a composition for diagnosing SARS coronavirus 2 infection, a method for detecting SARS coronavirus 2, and a SARS coronavirus 2 infection diagnostic kit, wherein a receptor and an antibody that binds to SARS coronavirus 2 spike protein are used in order to detect SARS coronavirus 2 (SARS-CoV-2) or diagnose SARS coronavirus 2 infection (COVID-19).

### BACKGROUND

### Technical Field

The present invention relates to a diagnostic kit using a receptor and antibody that binds to SARS coronavirus 2 spike protein for effective detection or diagnosis of SARS coronavirus 2 (SARS-CoV-2) spike protein.

### Background Art

As the gene sequence of SARS coronavirus 2, which was first reported in Wuhan, China and spread to epidemic, was released, WHO released the molecular genetic diagnosis method for SARS-CoV-2 on its website.
(https://www.who.int/emergencies/diseases/novel-coronavirus-2019/technical-guidance).

SARS-CoV-2 is an RNA virus belonging to Coronaviridae, classified as a class 1 infectious disease novel infectious disease syndrome. So far, it has been spread through droplets (saliva) and contact, in particular, it is known that transmission is possible through droplets (saliva) produced when coughing or sneezing, and by touching objects contaminated with SARS coronavirus 2 and then touching your eyes, nose, and mouth. It is usually known with an incubation period of 1 to 14 days, with an average incubation period of about 4 to 7 days. The main symptoms include fever, malaise, cough, shortness of breath, and pneumonia, and various respiratory infections ranging from mild to severe, along with sputum, sore throat, headache, hemoptysis, nausea, and diarrhea, and to treat this, symptomatic treatments such as fluid supplementation and antipyretics and general-purpose antiviral agents are being administered in clinical practice, but there is no specific anti-barrier agent. In order to diagnose SARS-CoV-2 infection, the virus is isolated from an upper or lower respiratory tract sample and infection is diagnosed through real-time gene amplification of specific genes.

First discovered in the 1960s, the name of the coronavirus is derived from the Latin word corona, meaning crown, because of its unique shape of the spike protein. When infected with humans, it causes common cold symptoms, but SARS (2003) and MERS (2012), which are mutated viruses, caused pneumonia and severe acute respiratory syndrome, leading to death with a high mortality rate. SARS coronavirus 2 is the most recently identified strain of coronavirus, whose genetic structure is 79.5% identical to SARS virus and 96% identical to the bat coronavirus.

SARS coronavirus 2, like the existing SARS coronavirus, mainly infects the human body through the oral mucosa and lungs. The reason is that a receptor called angiotensin-converting enzyme 2 (ACE2) and SARS coronavirus 2 spike protein must bind to enter into human host cells. ACE2 is an enzyme that acts on heart function and blood pressure regulation and is found in the heart, kidneys, gastrointestinal mucosa, or lungs, and among them, is known to have a higher rate of infection through the respiratory tract than the internal organs that have to travel through the bloodstream. As a result of studying how SARS-CoV-2 penetrates human cells, a joint research team at West Lake Advanced Research Institute in China (WIAS) and Tsinghua University succeeded in identifying the binding mechanism between the ACE2 receptor and SARS-CoV-2 spike protein. In addition, the joint research team at the American Institute of Allergy and Infectious Diseases Vaccine Research Center at the University of Texas in the U.S., using Cryo-EM technology, similarly to the Chinese joint research team, reported that SARS-CoV-2 spike protein has much higher binding affinity to the ACE2 receptor than the existing SARS-CoV-2 spike protein. This difference in avidity may be one clue explaining the high infectivity of SARS-CoV-2 because it binds better to ACE2, a receptor on human host cells.

Existing virus detection uses molecular biological methods such as PCR (gene amplification), the PCR technique has high sensitivity because it is amplified exponentially in proportion to time, but it is difficult to apply to the field because it requires specialized pre-treatment and requires complex and specialized experiments and equipment.

Currently, Virus infection is diagnosed through real-time RT-PCR using primers and/or probes that specifically bind to cDNA obtained by extracting viral RNA from a sample from a patient infected with SARS-CoV-2 in the laboratory and reverse transcribed to DNA. However, such real-time gene amplification has a disadvantage in that it is difficult to utilize it for rapid point-of-care.

Currently, a diagnostic method for detecting antibodies through a serological test used for SARS coronavirus 2 diagnostic test has been developed, but this is to detect the formation of IgM and IgG antibodies in the blood of an infected patient, and it is difficult to diagnose because antibodies are not formed at the initial stage of infection.

Although the enzyme immunoassay method was developed for the diagnosis of SARS coronavirus 2 antigen, this requires several processes, takes a lot of time, and requires expensive equipment, so it is difficult to make a simple and quick diagnosis, and it has disadvantages in that it is more expensive than a rapid kit. Meanwhile, the immunoassay using the lateral flow assay is a method of reading nanoparticles or fluorescence that can visually identify the presence or absence of antigen-antibody reaction, and enables rapid diagnosis and has the advantage of being inexpensive compared to enzyme immunoassay. However, the rapid test method using the existing lateral flow assay has disadvantages in that it takes a lot of time and money to develop an antibody, such as requiring 2 types of antibody pairs that bind to an antigen.

Therefore, in the present invention, it was attempted to develop a rapid diagnostic technology using a lateral flow assay using a pair between a receptor and an antibody instead of 2 types of antibody pairs that bind to an existing antigen.

### DETAILED DESCRIPTION OF THE INVENTION

### Technical Problem

The present invention has been derived from the above needs, and the present inventors completed the present invention by confirming that the receptor and antibody binding to SARS coronavirus 2 spike protein are effective for detection or diagnosis of SARS coronavirus 2 spike protein.

### Technical Solution

In order to solve the above problem, the present invention provides a composition for detecting SARS-CoV-2 comprising a receptor binding to SARS coronavirus 2 spike protein; an antibody capable of pairing with the receptor and binding to SARS coronavirus 2 spike protein; wherein the antibody is conjugated to a visibly identifiable nanostructure, or the secondary antibody recognizing the antibody is conjugated to a visibly identifiable nanostructure.

Also, the present invention provides a composition for diagnosing SARS coronavirus 2 infection comprising a receptor binding to SARS coronavirus 2 spike protein; an antibody capable of pairing with the receptor and binding to SARS coronavirus 2 spike protein; wherein the antibody is conjugated to a visibly identifiable nanostructure, or the secondary antibody recognizing the antibody is conjugated to a visibly identifiable nanostructure.

In another example, the present invention provides a method for detecting SARS coronavirus 2 comprising:

a step of incubating the composition for detecting SARS coronavirus 2 according to one aspect of the present invention and a separated sample;

a step of incubating a sample that does not contain an antigen as a control;

a step of incubating for 20 minutes; and

a step of analyzing the signal from the detection point;

Also, the present invention provides a kit for diagnosing SARS-CoV-2 infection, including a composition for detecting SARS-CoV-2, and instructions for use, comprising a receptor binding to SARS coronavirus 2 spike protein; an antibody capable of pairing with the receptor and binding to SARS coronavirus 2 spike protein; wherein the antibody is conjugated to a visibly identifiable nanostructure, or the secondary antibody recognizing the antibody is conjugated to a visibly identifiable nanostructure.

### Effects of the Invention

The present invention relates to a diagnostic kit comprising a receptor and an antibody that binds to SARS coronavirus 2 spike protein developed by replacing the immunodiagnostic method using a capture antibody and a detection antibody as a representative method for detecting an existing antigenic protein, and can be usefully used for detecting SARS-CoV-2 or diagnosing SARS-CoV-2 infection.

### BRIEF DESCRIPTION OF THE DRAWINGS

FIG. 1 shows an intracellular receptor (ACE2)-based LFIA according to an embodiment of the present invention.

a) Schematic diagram of ACE2 receptor recognition by SARS coronavirus 2. ACE2 is a cellular receptor for SARS coronavirus 2 as a type 1 membrane protein expressed in lung, heart, kidney and intestine.

b) Schematic diagram of an E2-based LFIA consisting of a sample pad, bonding pad, nitrocellulose membrane and absorbent pad. The test line located on the nitrocellulose membrane contains ACE2 for detection of SARS coronavirus 2 spike antigen. Anti-IgG antibodies are used in control lines. The proposed LFIA can achieve sensitive and selective detection for SARS coronavirus 2 spike antigen within 20 minutes.

FIG. 2 shows the results of indirect ELISA for the spike antigen of three different coronaviruses (SARS coronavirus S1, SARS coronavirus 2 S1 and MERS S1) in one embodiment of the present invention.

a) The interaction between the S1 antigen and ACE2 was tested in serially diluted samples (concentrations ranged from 200 to 0.05 ng/mL). In addition, three other antibodies, CR3022 (black) (b), F26G19 (red) (c), and S1-mAb (orange) (d); were used at the same concentration to examine the interaction with the spike antigen.

FIG. 3 shows the bio-layer interference (BLI) results of ACE2, CR3022, F26G19 and S1-mAb to SARS coronavirus 2 S1 antigen according to an embodiment of the present invention. The dotted lines represent the response curve of the BLI measurement, and the solid lines represent the titration curves based on a 1:1 binding model. Binding kinetics was measured for 4 different concentrations of the S1 antigen.

FIG. 4 shows the results of identification of a sandwich pair for detection of SARS coronavirus 2 spike antigen according to an embodiment of the present invention.

a) Schematic diagram of LFIA using ACE2 as capture probe and sandwich assay results from paired antibodies (CR3022, F26G19 and S1mAb). SARS coronavirus 2 S1 antigen (50 ng) was used as a positive control, and a buffer containing no S1 antigen was used as a negative control. After 20 minutes, the strips were taken with a smartphone and the peak intensity was analyzed.

b) LFIA schematic diagram and sandwich analysis results when antibodies were used as capture probes.

c) Peak intensity of capture probe (Pc)-detection probe (PD) pairs. A total of 12 pairs of positive controls (50 ng S1 antigen) were tested and densities analyzed. The peak intensity was calculated by subtracting the background intensity of the strip from the average intensity of dots.

FIG. 5 shows the sensitivity and specificity of ACE2-based LFIA according to an embodiment of the present invention.

a) Results of ACE2-based LFIA for detection sensitivity of SARS coronavirus 2 S1 antigen. ACE2-based LFIAs were tested at serially diluted antigen concentrations (concentrations ranged from 500 to 5 ng/m L). After 20 minutes, LFIA strip was filmed with a smartphone. The intensity of the test and control lines was transformed into a peak histogram in the image analyzer.

b) Comparative analysis of capture selectivity: positive control—SARS coronavirus S1, negative control—SARS coronavirus S1, MERS S1 and buffer solution. The detection efficiency of ACE2-based LFIA was demonstrated using antigen samples at three different concentrations (1 μg/mL, 200 ng/mL, and 50 ng/mL).

c) Peak intensity bar graph for the test line.

After 20 minutes for sample flow, the intensity of the test line was measured with a portable line analyzer.

Inset) Detection intensity for 5 ng antigen per reaction in each control. The limit of detection (LOD) is determined as the mean value of the negative control plus three times the standard deviation. P-values: ns>0.05, *p≤0.05, **p≤0.01, ***p≤0.001.

FIG. 6 shows laboratory confirmation results of ACE2-based LFIA using clinical samples according to an embodiment of the present invention.

a) Schematic diagram of the COVID-19 test using ACE2-based LFIA.

A nasopharyngeal smear from a COVID-19 patient is placed in a UTM. 50 μL of UTM containing SARS-CoV-2 is mixed in the running buffer at a 1:1 (v/v) ratio, and 100 μL of the mixed solution is injected into LFIA device. After 20 minutes, the line intensity of LFIA strips is semi-quantified on a portable analyzer.

b) Results of ACE2-based LFIA on detection sensitivity of cultured SARS coronavirus 2.

Serially diluted virus concentrations (concentration ranges from 1.07×10⁸ copies/mL to 5.35×10⁶ copies/mL) were tested. After 20 minutes, LFIA strips were taken with a smartphone and scanned with an image analyzer. The line densities of the test and control lines were converted into peak histograms. Also, the intensity of the test line was measured with a portable line analyzer (I_{L}: line intensity of the test line). Additionally, human coronavirus (OC43) was used as a negative control.

c) A bar graph of intensity on a test line measured on a portable analyzer.

The limit of detection (LOD) is determined as the mean value of the negative control (0 copies/mL SARS coronavirus 2) plus three times the standard.

d) Laboratory validation of ACE2-based LFIA compared to RT-qPCR using clinical samples.

i) Nasopharyngeal smear samples from COVID-19 patients (n=4) and healthy individuals (n=4) were measured in both ACE2-based LFIA and RT-qPCR.

Sensitivity was determined by dividing the number of actual positive samples by the number of positive samples tested. Specificity was determined by dividing the number of actual negative samples by the number of negative samples tested.

ii) RT-qPCR results on the detection of SARS coronavirus 2 specific gene (Env gene). Ct values and relative viral load in clinical samples were evaluated.

e) ACE2-based LFIA results on laboratory validation using clinical samples of COVID-19 patients. 20 minutes after sample loading, the intensity of the test line of LFIA strip was measured with a portable line analyzer. The limit of detection (LOD) is determined as the mean value of the negative control (health control) plus three times the standard deviation.

### DETAILED DESCRIPTION

Hereinafter, preferred embodiments of the present invention will be described in detail. In addition, in the following description, many specific details such as specific components are shown, which are provided to help a more general understanding of the present invention, and it will be apparent to those skilled in the art that the present invention may be practiced without these specific details. And, in describing the present invention, if it is determined that a detailed description of a related known function or configuration may unnecessarily obscure the gist of the present invention, the detailed description thereof will be omitted.

In order to achieve the object of the present invention, the present invention provides a composition for detecting SARS-CoV-2 comprising a receptor binding to SARS coronavirus 2 spike protein; an antibody capable of pairing with the receptor and binding to SARS coronavirus 2 spike protein; wherein the antibody is conjugated to a visibly identifiable nanostructure, or the secondary antibody recognizing the antibody is conjugated to a visibly identifiable nanostructure.

The present invention utilizes the binding between the receptor expressed on the surface of a host cell and SARS-CoV-2 spike protein, and means the skills that can detect or diagnose SARS-CoV-2 spike protein using a pair between the receptor and the antibody binding to the spike protein.

The receptor binding to SARS coronavirus 2 spike protein according to an embodiment of the present invention includes angiotensin converting enzyme 2 (ACE2), a lysate of a cell line expressing or overexpressing ACE2, and preferably an ACE2 protein, but is not limited thereto.

The antibody against SARS coronavirus 2 spike protein according to an embodiment of the present invention is a SARS coronavirus 2 spike protein antibody capable of pairing with ACE2, and it may include a complete antibody, an antigen-binding fragment of an antibody molecule, a synthetic antibody, a recombinant antibody, or an antibody hybrid. Preferably, it comprises a monoclonal antibody or a polyclonal antibody, and more preferably, the antibody binding to SARS coronavirus 2 spike protein comprises any one selected from the group consisting of the antibodies that recognize any one of S1, RBD (Receptor binding domain) and RBM (Receptor binding motif) as an antigen, but is not limited thereto.

The nanostructure according to an embodiment of the present invention is a visibly identifiable nanostructure and is a nanostructure bonded to a visibly identifiable dye, and preferably includes, but is not limited to, cellulose nanobeads or gold nanoparticles to which a visibly identifiable dye is bound.

The visually identifiable dye has a label that generates a detectable signal as a detection antibody or a secondary antibody. The label may include chemicals (e.g., biotin), enzymes (alkaline phosphatase, β-galactosidase, horse radish peroxidase and cytochrome P450), radioactive substances (e.g., C14, I125, P32 and S35), a fluorescent material (e.g., fluorescein), a light emitting material, a chemiluminescent and fluorescence resonance energy transfer (FRET).

The sample may be a biological material derived from a subject. The subject may be a vertebrate. The vertebrate may be a mammal. The mammal may be a primate including humans and non-human primates, a camel, or a rodent including mice and rats. The sample may be stored frozen or left in a natural state. The sample may be a water sample, a soil sample, a food sample, an air sample, a nasal swab sample, a nasopharyngeal wash, a branchioalveolar lavage, or a pleural fluid. The biological material may include nasal swap, nasal aspirate, nasopharyngeal swab, nasopharyngeal aspirate, blood or blood constituent, bodily fluid, saliva, sputum, or a combination thereof.

The present invention provides a composition for diagnosing SARS coronavirus 2 infection comprising a receptor binding to SARS coronavirus 2 spike protein; an antibody capable of pairing with the receptor and binding to SARS coronavirus 2 spike protein; wherein the antibody is conjugated to a visibly identifiable nanostructure, or the secondary antibody recognizing the antibody is conjugated to a visibly identifiable nanostructure.

The present invention provides a composition for diagnosing SARS-CoV-2 infection (COVID-19) in various individuals that can be infected with SARS-CoV-2. SARS-CoV-2 infection may include flu, cold, sore throat, bronchitis, or pneumonia, but is not limited thereto as long as it is a disease caused by SARS-CoV-2 infection.

Also, the present invention provides a method for detecting SARS coronavirus 2 comprising:

a step of incubating the composition for detecting SARS coronavirus 2 according to one aspect of the present invention and a separated sample;

a step of incubating a sample that does not contain an antigen as a control;

a step of incubating for 20 minutes; and

a step of analyzing the signal from the detection point;

Also, the present invention provides a kit for diagnosing SARS-CoV-2 infection, comprising a composition for detecting SARS-CoV-2 and instructions for use, comprising a receptor binding to SARS coronavirus 2 spike protein; an antibody capable of pairing with the receptor and binding to SARS coronavirus 2 spike protein; wherein the antibody is conjugated to a visibly identifiable nanostructure, or the secondary antibody recognizing the antibody is conjugated to a visibly identifiable nanostructure.

In the diagnostic kit of the present invention, the receptor binding to SARS coronavirus 2 spike protein may be used in a state immobilized on a solid support. Various materials may be used as the solid support, and examples thereof include, but are not limited to, cellulose, nitrocellulose, polyvinyl chloride, silica gel, polystyrene, nylon, activated beads, and the like.

Such a diagnostic kit may further include tools, reagents, and the like generally used for immunological analysis in the field of the present invention. Such tools or reagents include, but are not limited to, suitable carriers, labels capable of generating a detectable signal, solubilizing agents, detergents, buffers, stabilizers, and the like.

In one embodiment of the present invention, a “kit” is a diagnostic kit for detecting SARS coronavirus 2 comprising a sample pad, that is a site to be applied a liquid sample containing an analyte, a conjugate pad on which a visibly identifiable dye-bound nanostructure is movably supported, wherein the visibly identifiable dye-bound nanostructure is bound with an antibody or fragment thereof that binds to the SARS coronavirus 2 spike protein, and a visibly identifiable dye, and a chromatography membrane material is fluidly connected to the conjugate pad and the liquid sample moves by capillary movement, and comprises a capture region in which a receptor or fragment thereof that binds to the SARS coronavirus 2 spike protein is non-diffusively immobilized on the downstream of the conjugate pad; an absorbent pad in fluid communication with the chromatography membrane material; and the sample pad; bonding pad; a chromatographic membrane material and a solid support supporting the absorbent pad.

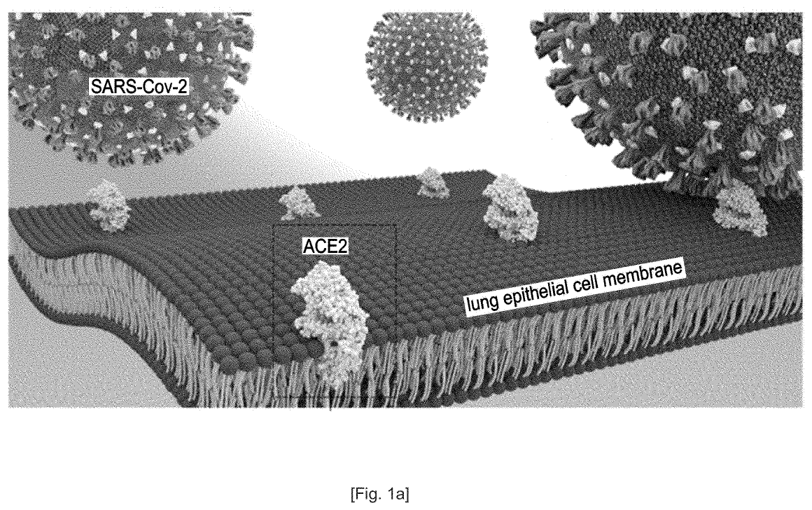

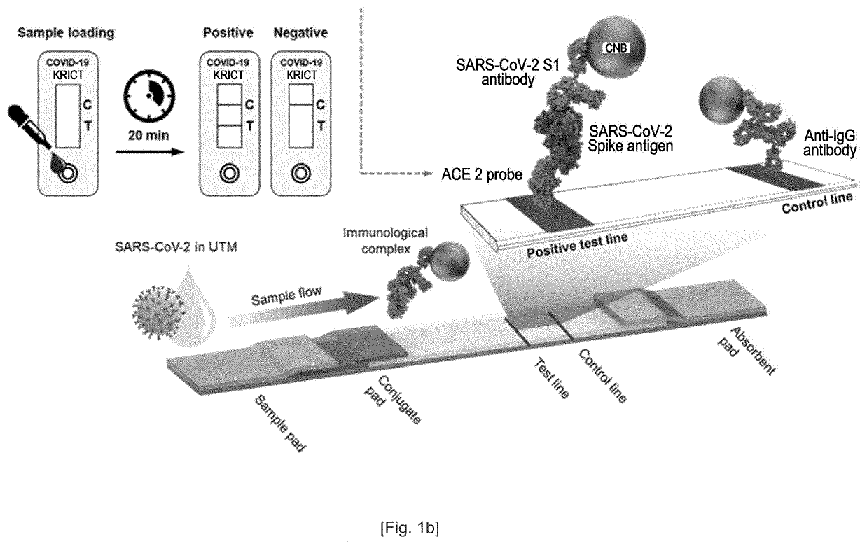

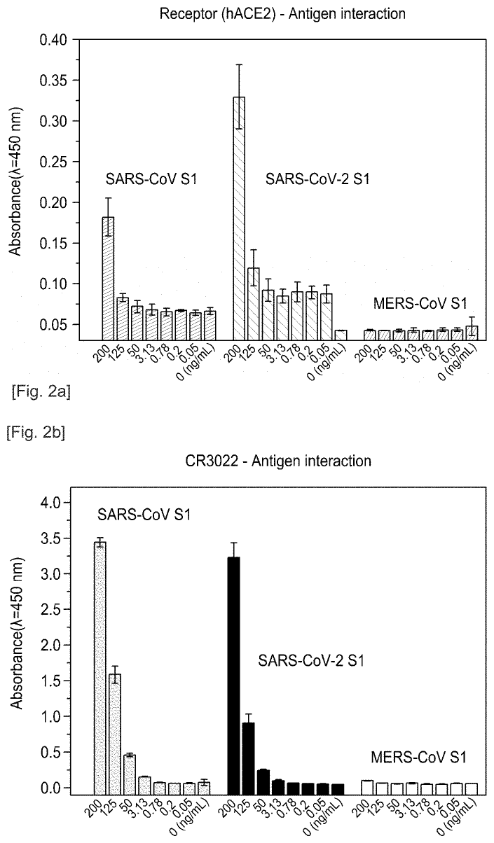

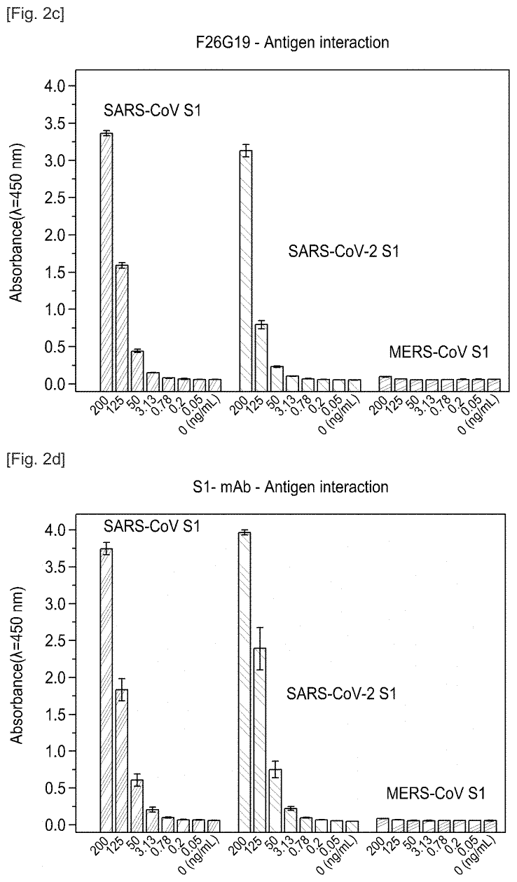

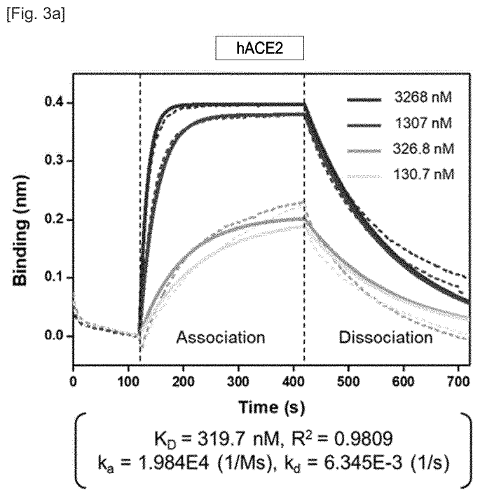

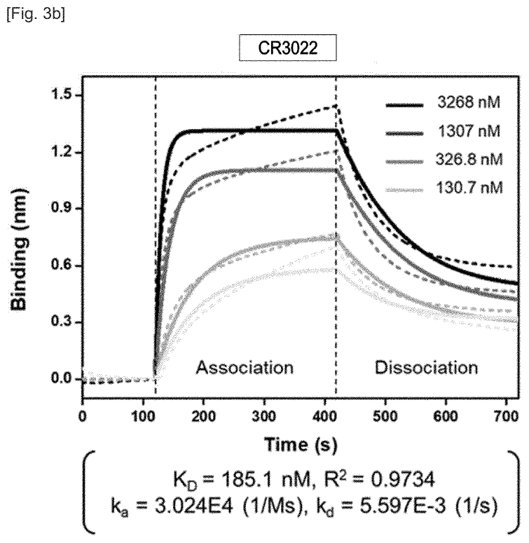

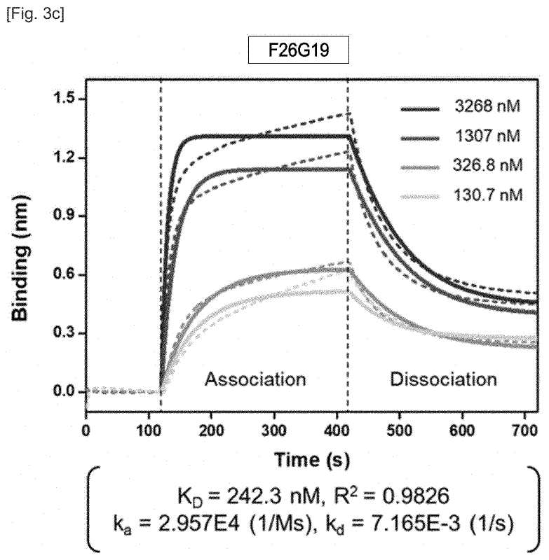

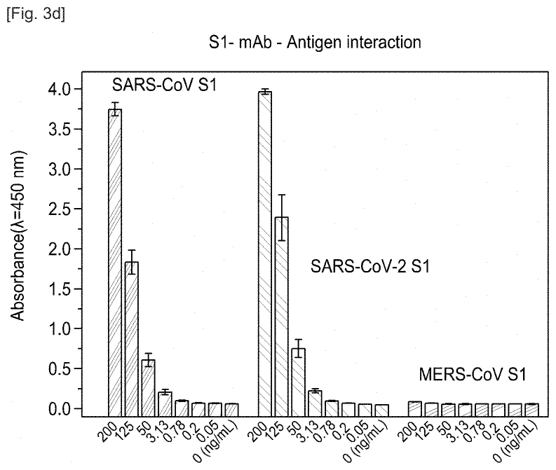

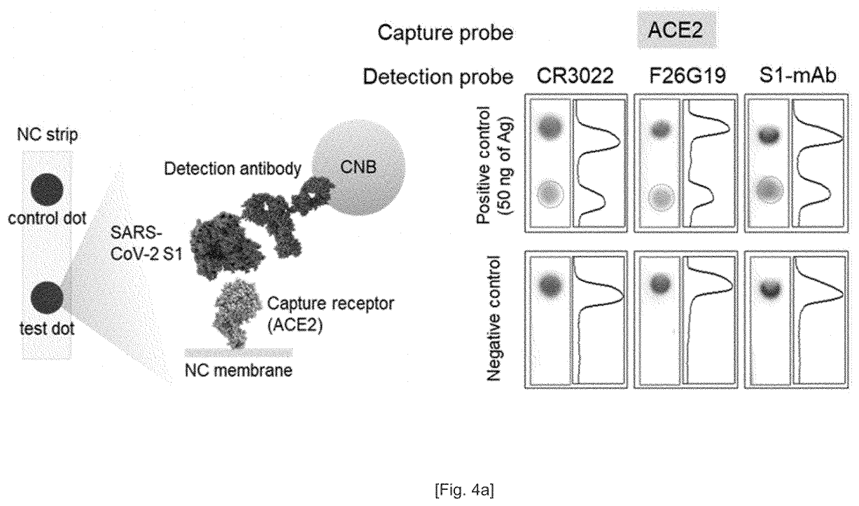

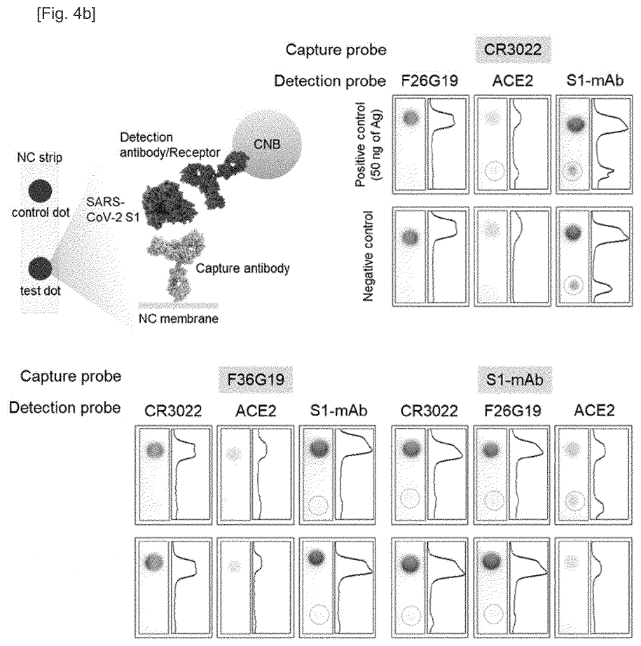

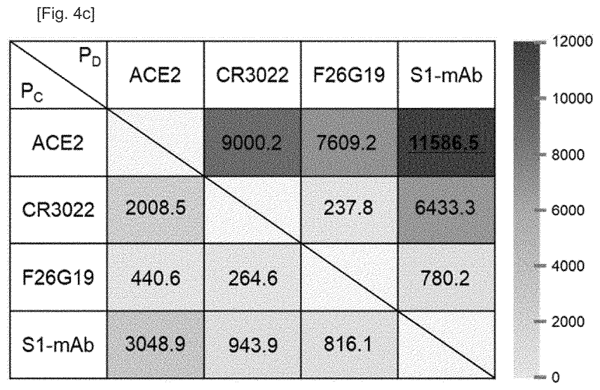

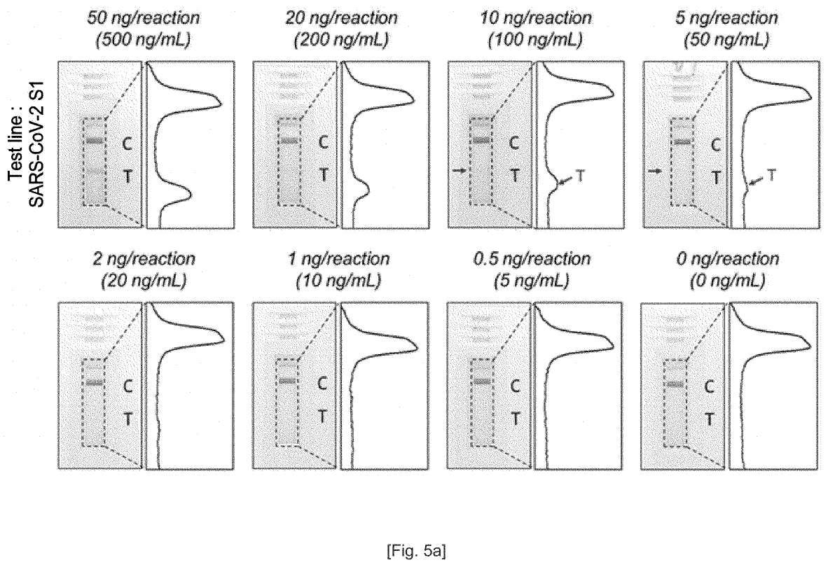

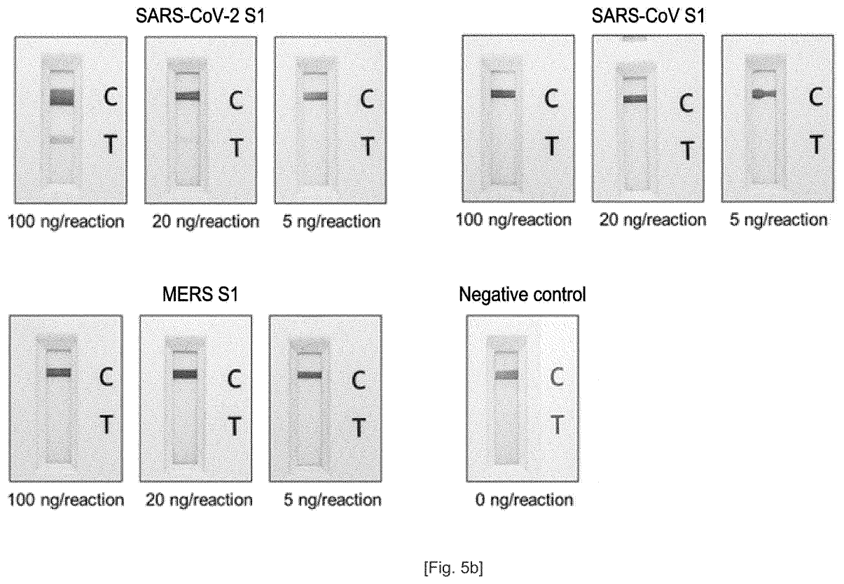

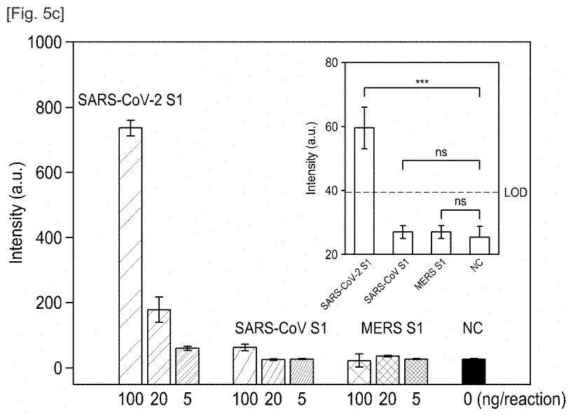

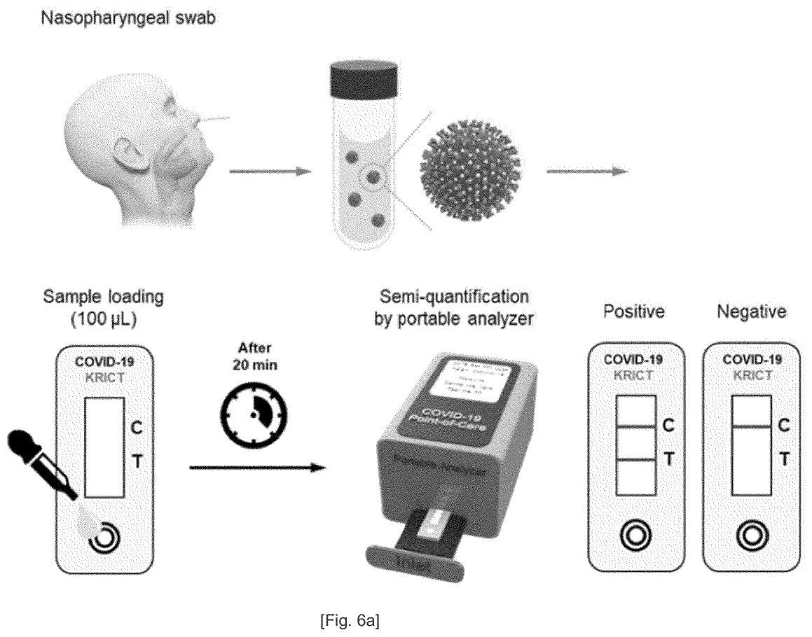

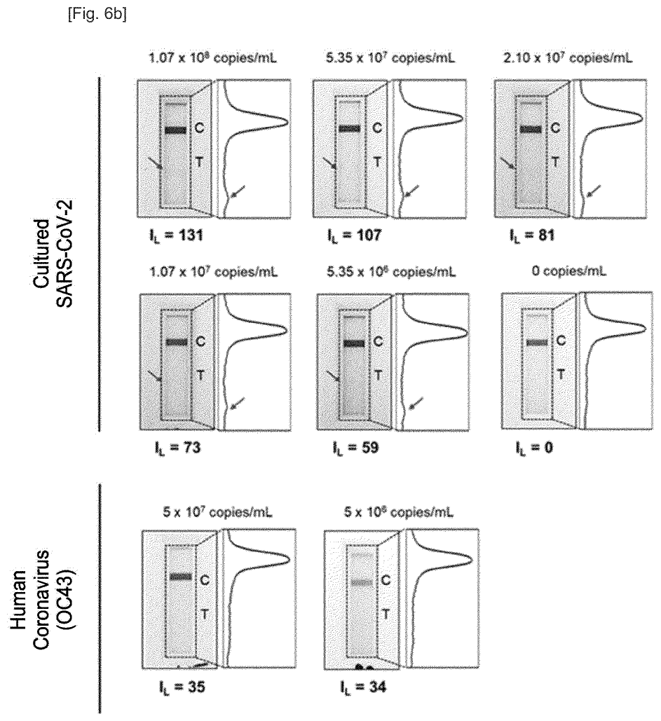

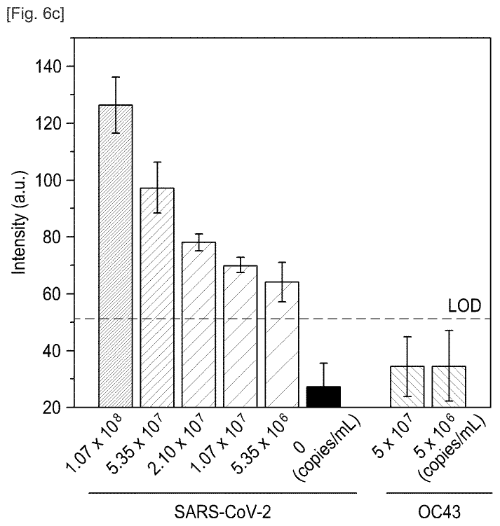

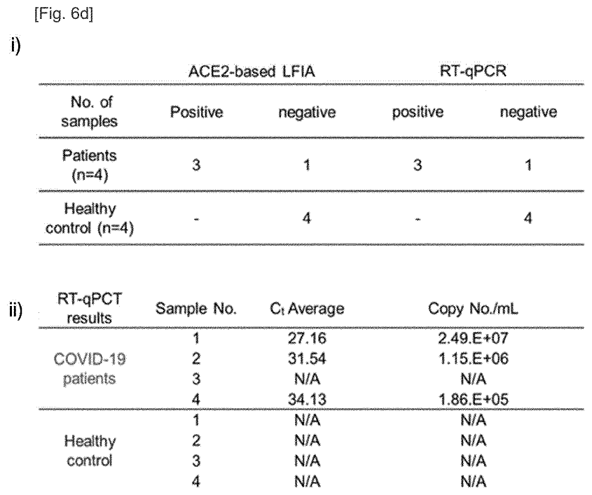

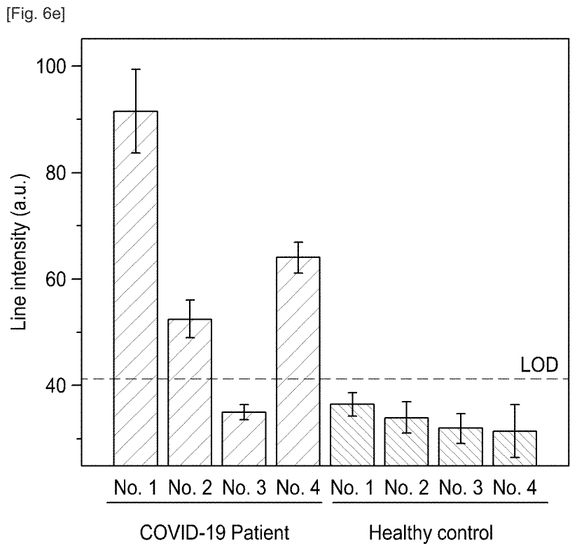

FIG. 1b is a schematic diagram illustrating an example of a diagnostic kit for detecting or diagnosing SARS coronavirus 2 or SARS coronavirus 2 spike protein of the present invention.

The kit comprises, on a solid support, supported by a test line (T) on which a receptor capable of binding to SARS coronavirus 2 spike protein is immobilized, and a control line (C) on which the chromatography material membrane is supported, wherein a sample pad, a conjugate pad supported by a nanostructure that binds an antibody binding to the SARS coronavirus 2 spike protein and a visually identifiable dye, and absorbent pad are overlapped and connected. When the sample is added to the sample pad on the strip, moving to the conjugate pad by capillary action, the antigen in the sample binds to the nanostructure in which the antibody binding to SARS coronavirus 2 spike protein in the binding pad and the visibly identifiable dye are bound, and is subjected to capillary migration through the chromatography material membrane downstream in the direction of the absorbent pad. When the antigen-nanostructure conjugate reaches the test line (T), color is developed, and when the conjugate is not present, color is not developed. Also, when the conjugate of the antibody and the nanostructure together with the sample passes through the control line (C), combined with a specific antibody for the conjugate and develops color, thereby confirming whether the capillary movement is correct.

The kit may be an immunoassay kit using a porous material as a solid carrier of an immunochemical component such as an antigen or antibody. The sample pad, conjugate pad, chromatography membrane material and absorbent pad used in one embodiment of the present invention are materials having sufficient porosity and volume to accommodate and contain a liquid sample to be analyzed, for example, microporous membrane material. Examples of the microporous membrane material include nylon, cellulosic material, polysulfide, polyvinylidene difluoride, polyester, and glass fiber. A preferred example of the microporous membrane material is a nitrocellulose membrane. The analysis device may have the form of a strip.

The nanostructure in which the antibody used in the kit of one embodiment of the present invention is bonded to a visibly identifiable dye can be prepared by a method well known in the art. Advantages and features of the present invention, and methods for achieving them, will become apparent with reference to the embodiments described below in detail. However, the present invention is not limited to the embodiments disclosed below, but will be implemented in various different forms, only the present embodiments are provided so that the disclosure of the present invention is complete, and to completely inform those of ordinary skill in the art to which the present invention belongs, the scope of the invention, the invention is only defined by the scope of the claims.

### EXAMPLE 1

### Enzyme-Linked Immunosorbent Assay (ELISA)

Maxisorp immunoplates (ThermoFisher SCIENTIFIC, MA, USA) were coated overnight at 4° C. with various concentrations (200, 100, 50, 25, 12.5, 6.25, 3.12, 0 ng/mL) per well of SARS-CoV-2 S1 protein (S1 subunit, Cat. No. 40591-V08H, Sino Biological, Beijing, China) aliquoted in 100 μL. The immunoplates were blocked for 1 hour with blocking buffer (Cat. No. DS98200, Invitrogen, CA, USA), and then hACE2, CR3022, F26G19 or S1-mAb in 100 μL blocking buffer was dissolved and added to each well and reacted for 1 hour. After washing with wash buffer (Cat. No. WB01; Invitrogen), the bound receptor and antibodies were reacted with horseradish peroxidase (HRP)-conjugated anti-mouse IgG (1:2000, Cat. No. 31437; Invitrogen), anti-human IgG (1:2000; Cat. No. 31413; Invitrogen) or anti-rabbit IgG (1:2000; Cat. No. 31463; Invitrogen) detection antibody for 1 hour. After repeating the washing process and aliquoting 100 μL of TMB solution (TMB solution, Cat. No. SB01; Invitrogen), observe the color change in the immunoplate, STOP solution (1M HCl) is aliquoted to stop the reaction. Then, the absorbance of the sample was measured at 450 nm using a microplate reader (BioTek, VT, USA).

### EXAMPL 2

### Biolayer Interferometry (BLI)

Fc-tag-bound human ACE2 (ACE2, Cat. No. 10108-H05H) and SARS coronavirus 2 spike monoclonal antibody (S1-mAb, Cat. No. 40150-R007), SARS coronavirus 2 spike protein (S1 subunit, Cat. No. 40591-V08H) and SARS coronavirus 2 RBD (Cat. No. 40592-V08B) were purchased from Sino Biological. Plasmids encoding heavy and light chains of each antibody at a ratio of 1:6 were mixed and transiently cotransfected into 293-F cells using PEI reagent (PolyScience, PA, USA). Six days after infection, the supernatant was collected, and CR3022 and F26G19 were purified on protein A columns (GE Healthcare, IL, USA). Binding affinity between SARS coronavirus 2 spike antigen (S1 and RBD) and four different antibodies (ACE2, CR3022, F26G19, and S1-mAb) were analyzed by BLI on a BLItz instrument (ForteBio, CA, USA) with. After hydrating the biosensor (ForteBio, USA) in tertiary distilled water for 10 minutes, binding affinity was measured by biolayer interferometry followed five steps. [1. Initial baseline setting (30 seconds), 2. Antibody immobilization (300 sec), 3. Second baseline setting (120 sec), 4. Antibody-antigen binding (300 seconds), 5. Antibody-antigen dissociation (300 seconds)]. All procedures are performed using sample dilution buffer (0.02% Tween 20, 150 mM NaCl, and 1 mg/mL BSA in 10 mM PBS with 0.05% sodium azide, pH 7.4). In the sample loading step, each antibody (100 μg/mL) is immobilized through binding of protein A coated on the biosensor surface to the Fc domain of the antibody (or receptor). After setting the initial baseline, 4 different concentrations of antigen were reacted with the antibody-binding biosensor to confirm antibody-antigen binding. 4 types of binding curves were analyzed based on a 1:1 binding model, and binding constants were obtained based on this.

### EXAMPLE 3

### Antibody-Bound CNB Preparation

Red cellulose nanobeads (CNBs) 1% stock solution was purchased from Asahi Kasei Fibers Corporation (NanoAct, Cat. No. RE2AA; Miyazaki, Japan), and the average diameter of the beads was 345 nm. The CNB conjugation kit includes a conjugation buffer, a blocking buffer, and a wash buffer, and was purchased from DCN Diagnostics (CA, USA). SARS coronavirus 2 antigen-specific antibodies (CR3022, F26G19, S1 mAb) and binding protein (ACE2) were bound to the surface of CNB and the test was performed according to the manufacturer's instructions. Briefly, 0.12 mL of 0.5 mg/mL antibody was mixed with 0.60 mL of 1% CNB stock solution and 0.12 mL of conjugation buffer and reacted at 37° C. for 2 hours. Then, 7.2 mL of blocking buffer was mixed and reacted additionally at 37° C. for 1 hour. The solution was centrifuged (14,400×g, 20 minutes, 4° C.), and the pellet was resuspended in 7.2 mL of wash buffer. After centrifugation (14,400×g, 20 minutes, 4° C.), the washed pellet was resuspended in 0.5 mL of wash buffer. The final concentration of antibody-bound CNB was approximately 0.1%. The exact concentration of CNB was calculated by measuring the absorbance at 554 nm with UV-vis spectrophotometry (Synergy H1; BioTek).

### EXAMPLE 4

### LFIA Strip Manufacturing

As shown in FIG. 1b, LFIA strips consist of a sample pad (Ahlstrom, Helsinki, Finland), a conjugation pad (Ahlstrom, Helsinki, Finland), a nitrocellulose membrane (NC membrane), and an absorbent pad (Ahlstrom, Helsinki, Finland). Conjugation pads were treated with 0.1% Triton X-100 (Cat. No. T8787; Sigma-Aldrich, MO, USA) prior to CNB fixation. After complete drying, stabilization buffer containing 0.05% of antibody-binding CNB solution (10 mM 2-amino-2-methyl-1-propanol (pH 9.0), 0.5% BSA, 0.5% β-Lactose, 0.05% Triton X-100, and 0.05% sodium azide) was sprayed onto the conjugation pad, and the reaction was carried out at 37° C. in a vacuum dryer (FDU-1200, EYELA, Tokyo, Japan; JSVO-30T, JSR, Gongju, Korea) for 1 hour. A test line and a control line containing a capture probe were divided into the following conditions on a nitrocellulose membrane using a line dispenser (BTM Inc., Uiwang, Korea) (dispensing speed, 50 mm/s; dispensing rate, 1 uL/cm). A mixture [1:1:1 (v/v/v) anti-human IgG (Cat. No. 12136, Sigma-Aldrich)/anti-rabbit IgG antibody (Cat. No. R5506, Sigma-Aldrich)/anti-mouse IgG antibody (Cat. No. A4416, Sigma-Aldrich)] of ACE2 (1 mg/mL) and 0.5 mg/mL anti-immunoglobulin G antibody was used in the sample dilution buffer (Cat. No. ab154873; Abcam, Cambridge, UK) to form test lines and control lines, respectively. After line separation, the nitrocellulose membrane was dried at 3° C. for 1 hour. In order to reduce non-specific reactions between a detection probe and a capture probe in the test line, a blocking solution (10 mM 2-amino-2-methyl-1-propanol (pH 9.0), 0.5% BSA, 0.5% β-Lactose, 0.05% Triton X-100, 0.05% sodium azide) was applied to the nitrocellulose membrane in a vacuum dryer (37° C.) for 1 hour. Each component of LFIA strip was correctly assembled and cut to 38 mm wide and stored for single LFIA testing.

### EXAMPLE 5

### Dot-Blot Assay for the Discovery of Sandwich Pairs

12 capture probe-detection probe pairs were tested to find a suitable pair for detecting SARS coronavirus 2 spike antigen. The affinities of 4 different SARS coronavirus 2 S1 and RBD antibodies were confirmed by ELISA, Western blot and BLI. A mixture [1:1:1 (v/v/v) anti-human IgG/anti-rabbit IgG antibody/anti-mouse IgG antibody] of the capture probe (1 mg/mL) and 0.5 mg/mL anti-immunoglobulin G antibody was used to form test dots and control dots, respectively. The dots were immobilized by dropping 0.5 uL of test or control solution onto nitrocellulose membranes (Advanced Microdevices). The nitrocellulose membrane was reacted with a blocking solution (10 mM 2-amino-2-methyl-1-propanol (pH 9.0), 0.5% BSA, 0.5% β-lactose, 0.05% Triton X-100, 0.05% sodium azide) in a vacuum dryer (1 hour, 37° C.). LFIA strips for dot-blot analysis were prepared by the method presented above. For the comparative analysis of 12 pairs, 50 ng of the target antigen (SARS coronavirus 2 spike S1) was reacted with each capture probe in the running buffer [10 mM adenosine monophosphate (AMP, pH 9.0), 5 mM EDTA, 200 mM Urea, 1% Triton X-100, 0.5% Tween 20, 500 mM NaCl, 1% PEG (MW 200)] for 10 minutes at room temperature, then the mixed sample was loaded onto LFIA strip. After 20 minutes, a red signal indicating antigen detection was confirmed through direct observation (naked eye) and a smartphone camera. Additionally, all points were quantitatively analyzed using an image analyzer (Sapphire Biomolecular Imager, Azure Biosystems, CA, USA), and the relative intensity of each point was calculated based on the average intensity of points excluding the background.

### EXAMPLE 6

### Sensitivity and Specificity of LFIA Assays

For the development of LFIA with high sensitivity and specificity for the detection of SARS-CoV-2, the concentration of CNB, the concentration of immobilized ACE2, and the composition of the running buffer were optimized. Finally, 0.05% CNB for the detection probe, 1 mg/mL of ACE2 for the immobilized capture probe, and running buffer [10 mM AMP, (pH 9.0), 5 mM EDTA, 200 mM urea, 1% Triton X-100, 0.5% Tween 20, 500 mM NaCl, 1% PEG (MW 200)] were selected. SARS coronavirus 2 S1 and SARS coronavirus 2 RBD antigens were diluted in a sample dilution buffer (Cat. No. ab154873, Abcam) by serial dilution method, the diluted sample was mixed in the running buffer in a ratio of 1:9 (v/v). The final concentrations of the diluted samples ranged from 500 to 5 ng/mL. 100 uL of the running buffer containing each antigen concentration was dropped into the inside of LFIA device. In this system, the sample flows with capillary force along LFIA strip and first encounters the antibody (S1-mAb)-bound CNB. When the antigen of the sample binds to the CNB bound to the S1-mAb and an antigen-probe complex is formed, the detection probe (ACE2) pre-immobilized to the nitrocellulose membrane is diagnosed. The test and control lines appear red at 20 minutes after sample loading and are analyzed on the Sapphire Biomolecular Imager.

For specificity testing, 2 different corona-associated spike antigens (e.g., SARS coronavirus S1 and MERS S1 antigens) were prepared at three concentrations (100, 20 and 50 ng/mL). Three concentrations of each antigen were loaded into the LIFA device, and the line intensity was quantified with a portable line analyzer (Light-G; WellsBio, Seoul, Korea). The positive intensity in the test line was 50 or higher according to the manufacturer's instructions for the portable analyzer. All experiments were performed in triplicate, and the limit of detection in FIG. 5c was calculated as the mean value of the negative control plus three times the standard deviation.

### EXAMPLE 7

### Isolation and Copy Number Quantification of Human Coronavirus-OC43 (HCoV-OC43) and SARS Coronavirus 2

Virus of HCoV-OC43, RNA isolation of gamma radiation treated SARS coronavirus 2 (Cat. No. NR-52287, BEI Resources, VA, USA) was performed with the QIAamp Viral RNA Mini Kit (Qiagen, Hilden, Germany) according to the manufacturers instructions. By serial dilution of viral RNA, the nucleocapsid (N) gene of HCoV-OC43 (1.16×10¹²-10⁰ copies/μL) or the envelope (Env) gene of SARS coronavirus 2 (7.7×10⁶-10⁰ copies/μL) was targeted as standard, the isolated viral RNA was synthesized as complementary DNA, serial RT-qPCR was performed with the Luna® Universal Probe One-Step RT-qPCR Kit (New England BioLabs, MA, USA) according to the manufacturer's instructions.

Primers of the HCoV-OC43 gene:

Forward
5′-AGC AAC CAG GCT GAT GTC AAT ACC
Reverse
5′-AGC AGA CCT TCC TGA GCC TTC AAT

Primers of SARS coronavirus 2 Env gene:

*Forward
5′-ACA GGT ACG TTA ATA GTT AAT AGC GT
Reverse
5′-ATA TTG CAG TAC GCA CAC A

FAM (6-carboxyfluorescein) and BHQ-1 (Back Hole Quencher-1) labeled probes:

ACA CTA GCC ATC CTT ACT GCG CTT CG

The single-step RT-qPCR was set for reverse transcription under 55° C. for 10 min, amplification of 45 cycles under 95° C. for 10 s, and 60° C. for 30 s. The reaction was analyzed using the Bio-Rad CFX 96 Touch Real-Time PCR System (CA, USA).

### EXAMPLE 8

### Evaluation of Clinical Use of ACE2-Based LFIA

Serial dilutions were made in the running buffer of SARS coronavirus 2 treated with gamma radiation, and a cultured SARS coronavirus 2 sample of 1.07×10⁸ copies/mL to 5.35×10⁶ copies/mL was prepared. 100 μL of running buffer containing each virus concentration was dropped into LFIA apparatus. After 20 minutes, it was confirmed that the test line appeared red, and it was quantified with a LightG portable analyzer (I_{L}: line intensity). Additionally, the densities of test and control lines were converted to peak histograms using a Sapphire Biomolecular Imager. Human coronavirus-OC43 (HCoV-OC43) was tested as a negative control in RT-qPCR. Two different concentrations (5×10⁷ copies/mL, and 5×10⁶ copies/mL) were loaded into LFIA device. 20 minutes after sample loading, non-specific reactions in the test line were evaluated in the same manner. To evaluate the clinical potential of ACE2-based LFIA, nasopharyngeal smear samples from COVID-19 patients (n=4) and healthy subjects (n=4) were applied to ACE2-based LFIA. Nasopharyngeal smear samples in universal transport media (UTM) collected from COVI D-19 patients were provided by Jeonbuk National University Hospital (Korea). Nasopharyngeal smear samples from healthy subjects were purchased from LEE Biosolutions (Cat. No. 991-31-NC, MO, USA). A healthy nasopharyngeal smear sample was suspended in UTM (Cat. No. UTNFS-3B-1, Noble Bio, Korea) and used for LFIA test. 50 uL of UTM obtained from nasopharyngeal smears taken from COVID-19 patients and healthy subjects was mixed in the running buffer at a ratio of 1:1 (v/v) and loaded into LFIA device. After 20 minutes, the intensity of the test line was analyzed with a LightG portable analyzer. The limit of detection was determined as the mean value of the healthy control plus three times the standard deviations. RT-qPCR was also used to compare test efficiencies using clinical samples. A primer-probe set was used to detect SARS coronavirus 2 specific envelope gene (Env gene). The primer is as follows.

Primers of SARS coronavirus 2 Env gene:

Forward
5′-ACA GGT ACG TTA ATA GTT AAT AGC GT
Reverse
5′-ATA TTG CAG TAC GCA CAC A

FAM (6-carboxyfluorescein) and BHQ-1 (Back Hole Quencher-1) labeled probes:

ACA CTA GCC ATC CTT ACT GCG CTT CG

The single-step RT-qPCR was set for reverse transcription under 55° C. for 10 min, amplification of 45 cycles under 95° C. for 10 s, and 60° C. for 30 s. The reaction was analyzed using the Bio-Rad CFX 96 Touch Real-Time PCR System (CA, USA).

### TEST EXAMPLE 1

### Interaction of SARS Coronavirus 2 S1 Antigen and Human Cell Receptor (ACE2)

The surface spike (S) glycoprotein of SARS coronavirus is recognized by the ACE2 receptor, resulting in endocytosis. The S protein of SARS coronavirus 2 and SARS coronavirus are very similar (Amino acid sequence homology: −77%). In some studies, the S protein of SARS-CoV-2 is superior to SARS-CoV in reaction affinity with ACE2. The strong affinity between the target antigen and the antibody (or receptor) is essential for the development of a highly sensitive and accurate diagnostic platform for antigen detection as well as the development of therapeutic agents and vaccines. The interaction of SARS coronavirus 2 S1 protein with ACE2 was determined by detection of SARS coronavirus 2 S protein by Western blot analysis, indirect ELISA and BLI. The interaction of the receptor (or antibody) with SARS coronavirus 2 S1 protein was measured by Western blot. Anti-SARS coronavirus 2 antibodies (CR3022, F26G19, S1-mAb) and human FC labeled ACE2 receptor (ACE2-Fc) were shown to be able to detect SARS coronavirus 2 S1 protein. This interaction was confirmed by ELISA, and the result was that the ACE2 receptor binds more strongly to SARS coronavirus 2 S1 protein compared to SARS coronavirus S1 protein, but not the MERS coronavirus S1 protein.

In contrast, the commercial anti-SARS coronavirus 2 antibody showed similar affinity to SARS coronavirus S1 protein and SARS coronavirus 2 S1 protein. The limit of detection of the ACE2 receptors, CR3022, F26G19 and S1-mAb for SARS coronavirus 2 S1 protein in the ELISA were approximately 125, 3.13, 3.13 and 0.78 ng/mL. In addition, the limit of detection of the ACE2 receptors, CR3022, F26G19 and S1-mAb for SARS coronavirus 2 RBD were 3.13, 125, 0.05 and 0.05 ng/mL. As a result of using the BLI method to confirm the detailed binding kinetics of ACE2 and SARS-CoV-2 S1 protein, the affinity of ACE2 for two mutations of SARS-CoV-2 spike (S1 and RBD) protein was measured. Three different commercial antibodies (CR3022, F26G19 and S1-mAb) were used to confirm binding to SARS coronavirus 2 S1 in the BLI assay. BLI is a label-exclusion technique for measuring biomolecular interactions related to changes in interference patterns after binding. The end of the BLI biosensor was coded for protein A, which was used to detect an effective target antibody. Three different commercial antibodies (CR3022, F26G19 and S1-mAb) and ACE2-Fc were immobilized on the surface of the BLI biosensor through specific interactions with protein A. The interaction with these antibodies was tested with SARS coronavirus 2 S1 and RBD.

Representative real-time binding sensor grams (dotted lines) and their fitting curves (solid lines) for receptor (or antibody)-antigens are shown in FIG. 3. Binding kinetic analysis was performed at four different antigen concentrations, and kinetic constants were calculated from four curves based on a 1:1 binding model. The K_{D} values of ACE for S1 and RBD were 319.7 and 13.18 nM. The RBD of S1 is mainly in charge of engagement with a host cell receptor ACE2. The previous study demonstrated that each monomer of the trimeric S protein of SARS coronavirus binds to ACE. It is known that the proteolytically activated S protein of SARS coronavirus 2 by proprotein convertase furin has had a higher binding affinity to ACE2 through RBD. Therefore, in the present invention, since RBD can bind to ACE2 more effectively than S1, the K_{D} value of ACE2 is expected to be lower for RBD binding than for S1. This also indicates that targeting the monomer of SARS coronavirus 2 S1 may be more effective than targeting S1 protein due to antigen detection using an antibody pair with ACE2, and also the reason for not performing the detection of RBD itself.

On the other hand, the K_{D} values of CR3022, F26G19 and S1-mAb were respectively 185.1, 242.3 and 73.35 nM for S1 and 21.52, 17.99, and 12.42 nM for RBD. Among the antibodies, S1-mAb showed the highest affinity for SARS coronavirus 2 S1, consistent with ELISA result. CR3022 and F26G19 are neutralizing antibodies that target the RBD of SARS coronavirus. Although recent studies showed that these antibodies can respond to the RBD of SARS-coronavirus 2, the affinities for SARS coronavirus 2 S1 and RBD was lower than that of S1-mAb, which was prepared using SARS coronavirus 2 S1 as an immunizing antigen. In regard to binding of S1 of SARS-CoV-2, ACE2 had the highest K_{D} value. However, the affinity of ACE2 for SARS coronavirus 2 RBD was similar to those of other commercial antibodies, raising an important possibility of substituting a commercial antibody for ACE as a replacement for in diagnostic platforms for antigen detection. In the present invention, the K_{D} values show similar trends despite the different K_{D} values used in each experiment.

### TEST EXAMPLE 2

### Sandwich Pair for Detection of SARS Coronavirus 2 S1 Antigen

For successful detection of the target antigen, two types of probes, the capture probe and the detection probe, are required, which recognize different sites of a specific antigens. However, under unpredictable circumstances like the current COVID-19 epidemic, the development of two different antibody pairs can be too time-consuming and expensive, and therefore could not be applied to rapid diagnosis in the field with the purpose of preventing the spread of disease. To overcome this disadvantage of antibody production, the intracellular receptor, human ACE2, was used in the present invention, which indicates whose binding affinity for target antigen is comparable to that of the antibody instead of the antibody.

In the present invention, dot-blot analysis was used to discover the pair for sandwich antigen detection. ACE2 and three commercially available antibodies (CR3022, F26G19 and S1-mAb) were immobilized on a nitrocellulose membrane (NC membrane) as capture probes. Then, other proteins (antibodies or receptors) not used in the immobilization were conjugated with cellulose nanobeads (CNB) for signal generation as detection probes, and the detection performance was evaluated. As shown in FIGS. 4a and 4b, a total of 12 pairs were used for the detection of SARS coronavirus 2 S1 antigen, and non-specific interactions between capture probe and detection probe were assessed. Furthermore, the intensity of each dot was analyzed on an image scanner. When ACE2 was used as the capture probe, and sandwich detection of the S1 antigen was successfully achieved with all three different detection probes. This implies that the ACE binding site of the SARS coronavirus 2 S1 antigen does not overlap with the epitope of the antibody. Additionally, in the present invention, no false-positive signal was observed in the negative control (e.g., without antigen).

On the other hand, when antibody was used as a capture probe, a false-positive signal was found due to a non-specific interaction between the capture and detection antibodies. Notably, non-specific interactions occurred between S1-mAb and CR3022 and between S1-mAb and F26G19 (the red circles in the negative control in FIG. 4b). Additionally, the epitope of CR3022 appeared to overlap with the epitope of F26G19, resulting in a decrease in the signal of the test dot. The results of the present invention indicate that discovery of suitable antibody pairs for sandwich assay is complicated by several variables. The introduction of ACE2 as replacement for antibodies could accelerate the development of antigen diagnostic kits and facilitate effective management of outbreaks. Moreover, overall signals, both test and control dots, were diminished in the case of ACE2-conjugated CNB. Evaluation of 12 capture-detection probe pairs allowed selection of the most suitable pair for sensitive detection of SARS coronavirus-2 S1 antigen: capture probe, ACE2, and detection probe, S1-mAb.

### TEST EXAMPLE 3

### Sensitivity and Specificity of ACE2-Based LFIA for SARS Coronavirus 2 S1

The LFIA sensor strip consists of a sample pad, a conjugation pad, a nitrocellulose membrane, and an absorbent pad. The sample comprising the target antigen is introduced onto the sample pad and sequentially encountered to the CNB dried on the conjugation pad. The CNBs are coated with detection antibody (S1-mAb) through hydrophobic and/or electrostatic interactions. The detection antibody-coated CNB captures the target SARS coronavirus 2 S1 antigen and transfers it to the nitrocellulose membrane. The test line comprising the capture probe (ACE2) detects the SARS coronavirus 2 S1 antigen that was previously captured by the detection probe (S1-mAb-conjugated CNB), allowing sandwich detection of the SARS coronavirus 2 S1 antigen. Meanwhile, the control line serves to determine whether the sample has flowed through and the biomolecules on the conjugate pad is active. For this purpose, anti-IgG antibodies are used to capture all antibodies that were already conjugated with the CNB. The test line and a control line are formed on the nitrocellulose membrane using a line dispenser. As the detection of the target antigen on the test line of LFIA, the red signal from the CNB makes it possible to visually confirm whether the sample contains the target antigen or not. To avoid non-specific interactions between capture probe and the detection probe in the test line, the nitrocellulose membrane was appropriately treated with a blocking solution. Unbound detection probe passes through the nitrocellulose membrane and ultimately reaches the absorbent pad located at the end of the strip, which serves to maintain the capillary forces.

To demonstrate the detection capability of the ACE-2 based LFIA (ACE2-LFIA), in the present invention, the sensitivity analysis was performed using serially diluted samples of SARS coronavirus 2 specific antigen (S1 and RBD, concentration range is 5-500 ng/mL). Twenty minutes after sample loading, the window of LFIA device was photographed using a smartphone, and the intensity of the test line and control line was analyzed with an image scanner and analyzer that converts the line color intensity to signal peak. In the case of S1 detection, the detection signal was gradually decreased according to the dilution factor, and the signal was present even in the case of 5 ng antigen. Additionally, in the present invention, it was confirmed that the detection signal was higher for RBD than for S1 for all diluted samples, and the detection signal was detected by ACE2-LFIA even at 1 ng of RBD. Here, no false-positive signals were observed in negative control (e.g., the absence of target antigen). Cellulose nanobeads (CNB), a colorimetric label used in LFIA, have known to have higher sensitivity than colloidal gold. Hence, in the present invention, the detection sensitivities of colloidal gold and CNB were compared. Colloidal gold-based LFIA detected RBD antigen at a concentration of 20 ng, which showed lower sensitivity compared to CNB-based LFIA. Because CNB has higher color intensity and surface area than colloidal gold in the same volume, CNB-based LFIA exhibits approximately 10 times more sensitive than colloidal gold-based LFIA, consistent with recent studies.

Next, the present invention confirmed the detection sensitivity of ACE2-LFIA using the S1 antigen of other coronaviruses (SARS coronavirus and MERS coronavirus). Three different concentrations (100, 20 and 5 ng/reaction) of each S1 antigen were introduced into LFIA device, and the intensity of the test line was measured after 20 minutes using a portable line analyzer. The line intensity of each LFIA device was measured in less than 10 seconds, to confirm whether rapid and accurate diagnosis was possible in a point-of-care or laboratory settings. The SARS coronavirus 2 S1 (<5 ng antigen) and RBD (<1 ng antigen) were successfully detected by the LFIA device, even at low concentrations. The sensitivity difference between SARS coronavirus 2 S1 and RBD using ACE2-based LFIA was related to the K_{D} value of ACE2, and S1-mAb were lower for RBD than for S1. Meanwhile, the MERS coronavirus S1 antigen was not detected even at a relatively high concentration (100 ng antigen), however 100 ng antigen of SARS coronavirus S1 was slightly detected. This means that 100 ng antigen of SARS coronavirus S1 was not distinguished using the ACE2-LFIA. The intensity slightly increased at a high concentration of SARS coronavirus S1, however this indicates that the proposed ACE2-LFIA can distinguish SARS coronavirus 2 from other coronaviruses due to the significant difference in intensity. As shown in FIG. 5c, the detection intensity for the S1 antigen was antigen concentration of 5 ng from three different coronaviruses. The detection signal of SARS coronavirus 2 S1 was higher than the limit of detection (LOD, mean value of negative controls+3×standard deviation), whereas SARS coronavirus S1 and MERS coronavirus S1 were similar to negative controls. Thus, it was confirmed that the proposed ACE2-LFIA exhibited high specificity to SARS coronavirus 2 antigen without significant cross-reactivity from other coronaviruses.

### TEST EXAMPLE 4

### Laboratory Confirmation Using Clinical Samples

Cultured viral and clinical samples were used for the laboratory confirmation of ACE2-LFIA. Viral load of cultured SARS coronavirus 2 and human coronavirus OC43 was measured by quantitative RT-PCR, with the standard curve of the E and N genes, respectively. The limit of detection of ACE2-LFIA was 5.35×10⁶ copies/mL of cultured viral sample of SARS coronavirus 2, however there were no positive signals (>50) from the ACE2-LFIA test with cultured samples of human coronavirus OC43.

To determine viral load (copy/mL) in clinical samples. RT-qPCR analysis was performed with nasopharyngeal and nasal swabs from COVID-19 patients (n=4) and healthy individuals (n=4). Viral load in the nasopharyngeal swab of COVI D-19 patients were investigated with the standard curve for the E gene of SARS coronavirus 2; Patient 1 was 2.49×10⁷ copies/mL, Patient 2 was 1.15×10⁶ copies/mL, and Patient 4 was 1.86×10⁶copies/mL. No SARS coronavirus 2 RNA was detected in Patient 3. This result was also confirmed by other independent RT-qPCR analysis, and might be associated with the degradation of viral RNA in UTM. Laboratory confirmation of ACE2-LFIA with clinical specimens of COVID-19 patients showed three positive results from only clinical specimens, by RT-qPCR analysis. The limit of detection of ACE2-LFIA was 1.86×10⁵ copies/mL (Patient 4), but no positive signal was observed from ACE2-LFIA test with nasal swabs from healthy subjects (n=4). Therefore, ACE2-based LFIA test could be helpful in detecting the S1 antigen of SARS coronavirus 2 from COVID-19 patients. Further developments of ACE2-based LFIAs with more specific antibodies, aptamers, affimers or nanobodies will be needed for more sensitive and specific detection of the SARS coronavirus 2 S1 antigen.

What is claimed is:

1. A composition for detecting SARS-CoV-2 comprising a receptor binding to SARS coronavirus 2 spike protein, and an antibody capable of pairing with the receptor and binding to SARS coronavirus 2 spike protein, wherein the antibody is conjugated to a visibly identifiable nanostructure, or a secondary antibody recognizing the antibody is conjugated to a visibly identifiable nanostructure.

2. The composition for detecting SARS-CoV-2 according to claim 1, wherein the receptor binding to the SARS coronavirus 2 spike protein comprises at least one selected from the group consisting of angiotensin converting enzyme 2 (ACE2) and lysate of a cell line expressing or overexpressing ACE2.

3. The composition for detecting SARS-CoV-2 according to claim 1, wherein
the antibody binding to SARS coronavirus 2 spike protein comprises any one selected from the group consisting of the antibodies that recognize any one of S1, RBD (Receptor binding domain) and RBM (Receptor binding motif) as an antigen.

4. The composition for detecting SARS-CoV-2 according to claim 1, wherein the nanostructure comprises any one selected from the group consisting of cellulose nano beads and gold nanoparticles.

5. The composition for detecting SARS-CoV-2 according to claim 1, wherein a sample comprises at least one selected from the group consisting of soil, water, air, food, nasal swab, nasopharyngeal wash, branchioalveolar lavage, pleural fluid, nasal swap, nasal aspirate, nasopharyngeal swap, nasopharyngeal aspirate, blood, blood constituent, bodily fluid, saliva, sputum and combinations thereof.

6. A composition for diagnosing SARS coronavirus 2 infection comprising a receptor binding to SARS coronavirus 2 spike protein, an antibody capable of pairing with the receptor and binding to SARS coronavirus 2 spike protein, wherein the antibody is conjugated to a visibly identifiable nanostructure, or a secondary antibody recognizing the antibody is conjugated to a visibly identifiable nanostructure.

7. The composition for diagnosing SARS coronavirus 2 infection according to claim 6, wherein the receptor binding to the SARS coronavirus 2 spike protein comprises at least one selected from the group consisting of angiotensin converting enzyme 2 (ACE2) and lysate of a cell line expressing or overexpressing ACE2.

8. The composition for diagnosing SARS coronavirus 2 infection according to claim 6, wherein the antibody conjugating to SARS coronavirus 2 spike protein comprise any one selected from the group consisting of the antibodies that recognize any one of S1, RBD (Receptor binding domain) and RBM (Receptor binding motif) as an antigen.

9. The composition for diagnosing SARS coronavirus 2 infection according to claim 6, wherein the nanostructure comprises any one selected from the group consisting of cellulose nano beads and gold nanoparticles.

10. The composition for diagnosing SARS coronavirus 2 infection according to claim 6, wherein a sample comprises at least one selected from the group consisting of nasal swab, nasopharyngeal wash, bronchioalveolar lavage, pleural fluid, nasal swap, nasal aspirate, nasopharyngeal swap, nasopharyngeal aspirate, blood, blood constituent, bodily fluid, saliva, sputum and combinations thereof.

11. A method for detecting SARS coronavirus 2 comprising:
a step of incubating the composition for detecting SARS coronavirus 2 of claim 1 and s separated sample;
a step of incubating a sample that does not contain an antigen as a control;
a step of incubating for 20 minutes; and
a step of analyzing signals from detection points.

12. A kit for diagnosing SARS coronavirus 2 infection comprising the composition of claim 1 and instructions for use.
